(19) United States
(12) Patent Application Publication
(10) Pub. No.: US 2015/0279231 A1
(43) Pub. Date: Oct. 1, 2015

(54) METHOD AND SYSTEM FOR ASSESSING CONSISTENCY OF PERFORMANCE OF BIOMECHANICAL ACTIVITY

(71) Applicant: Vibrado Technologies, Inc., Sunnyvale, CA (US)

(72) Inventors: Cynthia Kuo, Mountain View, CA (US); Quinn A. Jacobson, Sunnyvale, CA (US)

(21) Appl. No.: 14/668,203

(22) Filed: Mar. 25, 2015

Related U.S. Application Data
(60) Provisional application No. 61/970,149, filed on Mar. 25, 2014.

(51) Int. Cl. G09B 19/00; A61B 5/00; G06F 17/10

(52) U.S. Cl. CPC G09B 19/003; G06F 17/10; A61B 5/6801

(57) ABSTRACT

Sensors can be used to monitor repeated performances of a biomechanical activity. Data from the sensors are used to determine, for each performance of the biomechanical activity, values or measurements of parameters that quantify various aspects of the biomechanical activity. A consistency metric, which represents biomechanical similarity of the multiple performances of the biomechanical activity, is obtained from the parameter values that were derived from the sensor data. The consistency metric may be used to provide a quantitative assessment of consistency of performance of the biomechanical activity. This can be useful in athletic training as well as in physical therapy and rehabilitation.

### CROSS-REFERENCE TO RELATED APPLICATION

This application claims the benefit of U.S. Provisional Application No. 61/970,149, filed Mar. 25, 2014, which is incorporated herein by reference in its entirety and for all purposes.

### FIELD

The invention relates, in general to equipment for evaluating biomechanical activity, and more particularly to a method and system for assessing consistency of performance of the biomechanical activity.

### BACKGROUND

Consistent performance is the foundation of a successful competitor. In any type of competition, such as in in sports, music, etc., high-level competitors are all capable of flawless execution. Many are able to perform flawlessly during practice. However, winners and champions are able to perform flawlessly in competition. The hallmark of success is consistent performance, across all situations and environments.

As Vince Lombardi presumably said, “Practice does not make perfect. Only perfect practice makes perfect.” Perfect practice is not about going through the motions for hours on end. It means practicing with deliberation: challenging yourself to try something just beyond your current ability, analyzing your performance, and correcting any mistakes. This cycle is repeated continuously. Perfect practice makes training time more efficient and effective; it reinforces the muscle memory required to execute a skill flawlessly and consistently.

Conventionally, athletic consistency is generally a qualitative evaluation by human eye, either in person, by reviewing video recordings, or using an advanced motion caption system in a laboratory.

What is needed is a method and system capable of quantitative evaluation of consistency. A metric that assesses the biomechanical similarity of a repeated movement would be useful for athletic training, as well as physical therapy and rehabilitation. In sports, this consistency metric would be useful for developing and refining an athletic skill. In physical therapy and rehabilitation, a consistency metric would be useful for learning, correcting, strengthening, or refining the movements of patients.

### SUMMARY

Briefly and in general terms, the present invention is directed to a method, system, and computer readable medium for assessing consistency of performance of a biomechanical activity.

In aspects of the invention, a method comprises receiving data from sensors configured to detect a biomechanical activity of a body, the data representing multiple performances of the biomechanical activity. The method also comprises, for each performance of the biomechanical activity, determining a value for each one of a plurality of parameters that quantify various aspects of the biomechanical activity, the value being determined from the received data. The method also comprises computing a consistency metric representing biomechanical similarity of the multiple performances of the biomechanical activity. The computing is performed using the values for the plurality of parameters from the multiple performances of the biomechanical activity.

In aspects of the invention, a system comprises means for receiving data from sensors configured to detect a biomechanical activity of a body, the data representing multiple performances of the biomechanical activity. The system also comprises means to determine, for each performance of the biomechanical activity, a value for each one of a plurality of parameters that quantify various aspects of the biomechanical activity, the value being determined from the received data. The system also comprises means for computing a consistency metric representing biomechanical similarity of the multiple performances of the biomechanical activity, the computing performed using the values for the plurality of parameters from the multiple performances of the biomechanical activity.

In aspects of the invention, a system comprises a plurality of sensors configured to detect a biomechanical activity of a body, the plurality of sensors configured to provide data on multiple performances of the biomechanical activity. The system further comprises a processor device configured to receive and use the data to determine, for each performance of the biomechanical activity, a value for each one of a plurality of parameters that quantify various aspects of the biomechanical activity. The processor device is further configured to use the values for the plurality of parameters from the multiple performances of the biomechanical activity to compute a consistency metric representing biomechanical similarity of the multiple performances of the biomechanical activity.

In aspects of the present invention, a non-transitory computer readable medium has a stored computer program embodying instructions, which when executed by a computer system, causes the computer system provide an assessment of consistency of performance of a biomechanical activity. The computer readable medium comprises instructions for receiving data from sensors configured to detect a biomechanical activity of a body, the data representing multiple performances of the biomechanical activity. The computer readable medium further comprises instructions to determine, for each performance of the biomechanical activity, a value for each one of a plurality of parameters that quantify various aspects of the biomechanical activity, the value being determined from the received data. The computer readable medium further comprises instructions for computing a consistency metric representing biomechanical similarity of the multiple performances of the biomechanical activity, the computing performed using the values for the plurality of parameters from the multiple performances of the biomechanical activity.

The features and advantages of the invention will be more readily understood from the following detailed description which should be read in conjunction with the accompanying drawings.

### BRIEF DESCRIPTION OF THE DRAWINGS

FIG. 1 is a schematic diagram showing an exemplary system for assessing consistency in repeated performances of a biomechanical activity performed by a human or animal subject.

FIG. 2 is a chart showing an exemplary stream of data from sensors of the system of FIG. 1, the data containing biomechanical and biometric information associated with the biomechanical activity.

FIG. 3 is a flow diagram showing an exemplary method for assessing consistency in repeated performances of a biomechanical activity performed by a human or animal subject.

FIG. 4 is a flow diagram showing an exemplary method for calibrating sensor data at the start of training or therapy sessions.

FIG. 5 is a photographic illustration showing an exemplary system for assessing consistency in repeated performances of a biomechanical activity.

FIG. 6 is a schematic diagram showing an exemplary processor device in a system for assessing consistency in repeated performances of a biomechanical activity.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### DETAILED DESCRIPTION OF EXEMPLARY EMBODIMENTS

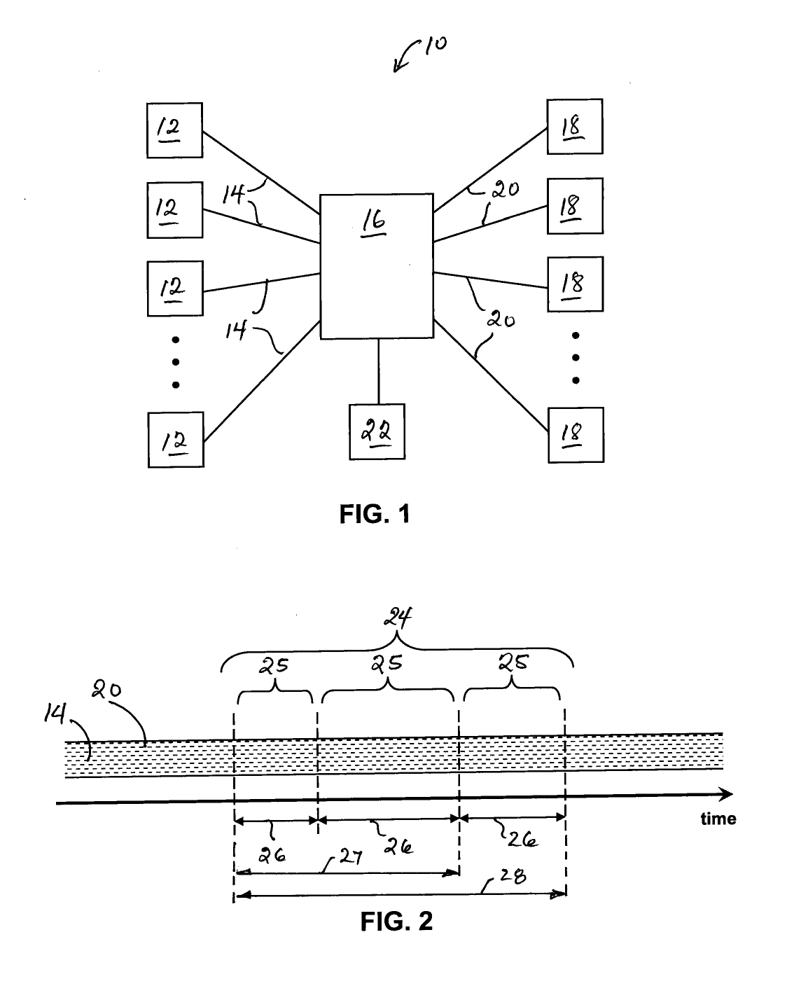

Referring now in more detail to the exemplary drawings for purposes of illustrating exemplary aspects of the invention, wherein like reference numerals designate corresponding or like elements among the several views, there is shown in FIG. 1 exemplary system 10 capable of quantitative evaluation of consistency of performance of a biomechanical activity. A biomechanical activity includes one or more biomechanical movements. A biomechanical activity can be relatively simple, such when moving a eating utensil from a plate to one's mouth, which may involve flexing the wrist joint to pick up food, then flexing the shoulder joint to lift the arm, and then followed by flexing the elbow joint to bring the utensil to one's mouth. A biomechanical activity can also be relatively complex, such as when pitching a baseball which involves a series of biomechanical movements involving the lower extremities, trunk, shoulder, elbow, wrist and hand.

System 10 comprises a plurality of sensors 12 configured to detect a biomechanical activity of the body of a subject and provide data 14 on multiple performances of the biomechanical activity. For example, data 14 is provided for repeated performances of the same biomechanical activity, such as during a practice session in which the athlete performs several basketball shots repeatedly. Although four sensors 12 are illustrated, system 10 may include a lesser or greater number of sensors 12 depending on the type of biomechanical activity to be evaluated and/or the physical condition of the human or animal subject.

Sensors 12 are communicatively coupled to processor device 16. As used herein, “communicatively coupled” means coupled in a way that enables transmission and/or receipt of data. For example and without limitation, devices that are communicatively coupled to each other can be configured to communicate with each other wirelessly through the air (e.g., via radio signals, ultrasonic signals, or optical signals) or through electrical or optical cables.

Processor device 16 is configured to receive and use data 14 to determine, for each performance of the biomechanical activity, a value for each one of a plurality of parameters. The parameter values quantify various aspects of the biomechanical activity.

When the biomechanical activity is a basketball shot, for example, the plurality of parameters for the shooting arm may include the orientation of the upper arm at the moment just before throwing the ball, the angle of the elbow joint at the moment just before throwing the ball, and the angle of the wrist joint at the moment just before throwing the ball. Additional parameters may characterize other aspects of the basketball shot, such as acceleration of the upper arm, acceleration of the forearm, and rate of rotation of the elbow joint when throwing the ball. Additional parameters may characterize other aspects of the basketball shot, such as orientation of the upper arm, angle of the elbow joint, and angle of the wrist joint after the ball has been released.

As indicated by the forgoing discussion and as shown in FIG. 2, a biomechanical activity 24 can be a series of discrete biomechanical movements 25. The horizontal line represents time, and the bar schematically represents the stream of data received by processor device 16 from the sensors. Additional parameters may characterize the timing of biomechanical movements. For example and without limitation, timing may correspond to the amount of time 26 to complete a biomechanical movement, time 27 between one biomechanical movement and another biomechanical movement, time 28 to complete the biomechanical activity, and the sequential order in which biomechanical movements were performed.

The invention is not limited to the foregoing examples for parameters. Many other parameters may be evaluated by processor device 16 for a basketball shot. Also, the parameters evaluated by processor device 16 will depend on the type of biomechanical activity, whether it be a golf swing, baseball pitch, other athletic skill, or activity performed during physical therapy and rehabilitation. For example and without limitation, the plurality of parameters may characterize biomechanical movements associated with the face, neck, back, abdomen, legs, and/or feet.

Processor device 16 is further configured to use the values for the plurality of parameters from the multiple performances of the biomechanical activity to compute a consistency metric. The consistency metric represents biomechanical similarity of the multiple performances of the biomechanical activity. Thus, the consistency metric allows a user of system 10 to quantitatively evaluate the degree to which the biomechanical activity was repeatedly performed with consistency. The user can be the person who is practicing the biomechanical activity, a coach, physical therapist, physician, or biomechanical specialist.

Sensors 12 are also referred to herein as motion capture sensors. Sensors 12 implement motion capture technology that enables system 10 to measure the nuances of the subject's movement. Sensors 12 may include inertial measurement units (IMUS) configured to be attached to the subject's body, such as on various locations on the arm, leg, torso, head, and/or other parts of the body. Exemplary IMUs are described further below.

Sensors which are described as being attached or capable of being attached to the subject's body can be attached in direct contact with the skin or attached to a garment, strap, shoe, glove, padding, or other item which is worn on and/or secured to the subject's body. The way in which the sensor is attached to the body will depend upon the type of sensor and its particular capabilities.

Additionally or alternatively, motion capture sensors 12 may include one or video cameras. Processor device 16 may use software algorithms which process video data 14 so that values for the plurality of parameters can be determined. Optionally, markers may be placed on the body of the human or animal subject to enable the software algorithm to measure biomechanical movements.

Additionally or alternatively, motion capture sensors 12 may include infrared projectors and infrared-capable image sensors. A non-limiting example is Kinect® available from Microsoft Corp. of Redmond, Wash.

Additionally or alternatively, motion capture sensors 12 may implement other technologies for motion tracking For example, sensors 12 may include transmitters that emit a magnetic field and a receiver that detects the magnetic field. Such technologies are available from Ascension Technology Corp. of Shelburne, Vt. and Polhemus of Colchester, Vt.

Optionally, system 10 comprises other types of sensors, such as sensors 18 configured to provide biometric data 20 on bodily conditions to processor device 16 before, during, and/or after one or more biomechanical movements of the biomechanical activity. Processor device 16 is communicatively coupled to sensors 18. Although four sensors 18 are illustrated, system 10 may include a lesser or greater number of sensors 18 depending on the type of biomechanical activity to be evaluated and/or the physical condition of the subject.

Sensors 18 are also referred to herein as biometric sensors. Any of the exemplary biometric sensors described herein may be attached or capable of being attached to the subject's body. The way in which the biometric sensor is attached to the body will depend upon the type of sensor and its particular capabilities.

Biometric sensors 18 may include myography sensors which enable processor device 16 to characterize muscle activity. Exemplary myography sensors are described further below.

Additionally or alternatively, biometric sensors 18 may include electrocardiography sensors that enable processor device 16 to characterize heart rate activity or heart rate variability of the subject. Thus, the plurality of parameters used by processor device 16 may include a parameter that defines heart rate activity or heart rate variability.

Additionally or alternatively, biometric sensors 18 may include galvanic skin response sensors that enable processor device 16 to characterize skin conductance of the subject. Thus, the plurality of parameters used by processor device 16 may include a parameter that defines skin conductance.

Additionally or alternatively, biometric sensors 18 may include sensors configured for pulse oximetry that enable processor device 16 to characterize oxygen saturation of the subject's blood. Thus, the plurality of parameters used by processor device 16 may include a parameter that defines blood oxygen saturation.

Additionally or alternatively, biometric sensors 18 may include respiration sensors that enable processor device 16 to characterize respiration rate of the subject. Thus, the plurality of parameters used by processor device 16 may include a parameter that defines respiration rate.

Additionally or alternatively, biometric sensors 18 may include sensors configured for electroencephalography (EEG) that enable processor device 16 to characterize neurological activity of the subject. Thus, the plurality of parameters used by processor device 16 may include a parameter that defines neurological activity.

Using biomechanical data 14 and/or biometric data 20, processor 16 can describe the execution of a biomechanical activity, such as an athletic skill or therapeutic/rehabilitative exercise, through a set of parameters. The set of parameters (i.e., the types of parameters which are to be measured) may be predefined, such as being stored in memory of processor device 16 or communicated to processor device 16 from device 22 (FIG. 1) which can be a database or computer device. The set of parameters may be predefined based on human expertise, such as the expertise of an athletic coach, physical therapist, and/or biomechanics specialist. For example, the set of parameters can be selected as those that capture the essential characteristics of the various biomechanical movements of the activity to be evaluated.

In addition or as alternatives to the parameters previously described, parameters may define any or a combination of: (a) biomechanical state of a body part in absolute three-dimensional space; (b) biomechanical state of a body part relative to another body part; (c) biomechanical state of a body part relative to an object, for example, a golf club or a basketball hoop; (d) timing of a biomechanical movement or series of biomechanical movements; (e) level of muscle activity before, during, and/or after a movement or series of biomechanical movements; (f) level of muscle fatigue before, during, and/or after a movement or series of biomechanical movements; and (g) other bodily conditions based on biometric data 20 before, during or after a movement or series of biomechanical movements. For parameter examples (a), (b) and (c), the phrase “biomechanical state” can mean physical orientation, such as direction or angle in which the body part is pointing or facing. Alternatively, the phrase “biomechanical state” can mean position, such as where the body part is located. Alternatively, “biomechanical state” can mean linear velocity or linear acceleration. Alternatively, “biomechanical state” can mean rotational velocity or rotational acceleration.

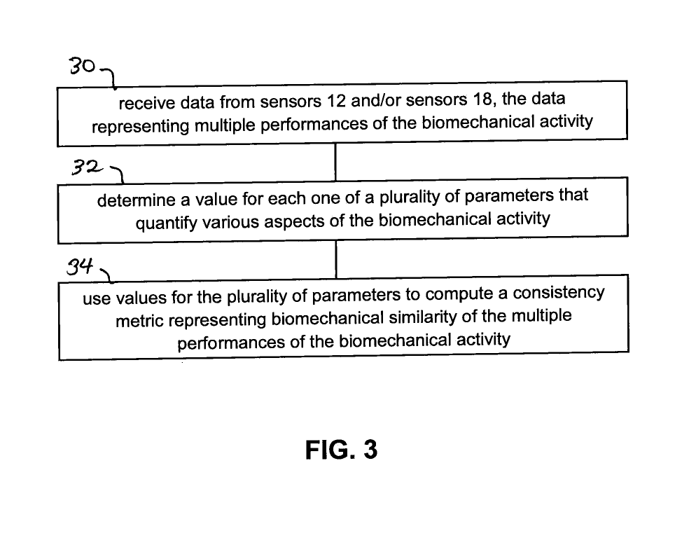

FIG. 3 depicts an exemplary method for assessing consistency of performance of a biomechanical activity of a human or animal subject. Although the method is described with reference to devices of system 10, it is to be understood that the method can be performed with other devices.

In block 30, processor device 16 receives data from sensors configured to detect a biomechanical activity of a body. The data represents multiple performances of the biomechanical activity. For example, a human or animal subject performs the same biomechanical activity several times, and it is desired to evaluate the degree to which the subject performed the biomechanical activity consistently. Data 14 from motion capture sensors 12 and/or data 20 from biometric sensors 18 are collected from each performance of the biomechanical activity.

Next in block 32, processor device 16 uses the data it receives to determine a value for each one of a plurality of parameters. Processor device 16 does this for each performance of the biomechanical activity. The parameter values quantify various aspects of the biomechanical activity.

For example, a first parameter (P₁) may define velocity of the right foot of the subject during a first biomechanical movement of the activity. A second parameter (P₂) may define the angle at the knee formed by the upper leg and lower leg at the start of a second biomechanical movement of the activity. A third parameter (P₃) may define the time between the first biomechanical movement and the second biomechanical movement. Processor device 16 may determine a value for P₁, P₂ and P₃ for each performance of the biomechanical activity as shown in TABLE I.

TABLE I

| | Performance of Biomechanical Activity | | | | Variation |
|---|---|---|---|---|---|
| | First | Second | Third | Fourth | vᵢ = σᵢ² |
| P₁ | 2.1 m/s | 2.4 m/s | 2.1 m/s | 2.1 m/s | 0.0169 |
| P₂ | 35 degrees | 37 degrees | 36 degrees | 35 degrees | 0.688 |
| P₃ | 0.5 second | 0.4 second | 0.5 second | 0.5 second | 0.00188 |

Next in block 32, processor device 16 computes a consistency metric that represents biomechanical similarity of the multiple performances of the biomechanical activity. To do so, processor device 16 uses the values for the plurality of parameters from the multiple performances of the biomechanical activity. In the example of TABLE I, processor device 16 computes a consistency metric using the four values for P₁, the four values for P₂ and the four values for P₃.

Processor device 16 may compute variation vᵢ for each parameter Pᵢ. The letter i ranges from 1 to n, where the letter n represents the total number of parameters used by processor device 16 to evaluate consistency of performance of the biomechanical activity. As used herein, variation vᵢ is a quantitative indicator of variation of parameter values for parameter Pᵢ across multiple performances of the biomechanical activity. There are various types of quantitative indicators for variation of a set of measurements. Examples for variation vᵢ include without limitation, statistical variance (σᵢ²), standard deviation (σᵢ), a quantity that includes σᵢ², and a quantity that includes σᵢ. As discussed below, the consistency metric may include a sum of vᵢ for all parameters i=1 to n.

Methods for computing a statistical variance and standard deviation (for a sample of measurements or an entire population of measurements) are known to persons of ordinary skill in the art and need not be described in detail herein. Computing variance and standard deviation includes computing a sum of squares relative to a target value. The sum of squares is expressed as:

Σⱼ₌₁^{m}[(valueⱼ of Pᵢ)−(target for Pᵢ)]²

where j ranges from 1 to m, and m is a total number of values determined for a particular parameter Pᵢ. The valueⱼ of Pᵢ is determined by processor device 16 from biomechanical data 14 and/or biometric data 20. The letter m may be equal to a total number of performances of the biomechanical activity, so processor device 16 may compute a new value for Pᵢ for each performance and these parameter values are used to compute the sum of squares. The target for Pᵢ is the desired value for Pᵢ. The target for Pᵢ can be based on human expertise, such as the expertise of an athletic coach, physical therapist, and/or biomechanics specialist. For example, a coach may set the target for Pᵢ. The target for Pᵢ may be stored in memory of processor device 16 or communicated to processor device 16 from device 22 (FIG. 1).

From the discussion above, it will be apparent that the target for Pᵢ is not necessarily equal to the average of all values of Pᵢ. In some aspects, the target for Pᵢ is not the average of all values of Pᵢ. In some aspects, the target for Pᵢ is the average of all values of Pᵢ. For example, the target for Pᵢ can be the long term average of values for Pᵢ across many practice or therapy sessions.

Processor device 16 may compute the consistency metric by computing a first quantity (Aᵢ) for each of the plurality of parameters (Pᵢ). Again, i=1 to n and the letter n represents the total number of parameters used by processor device 16 to evaluate consistency of performance of the biomechanical activity. The total number (n) of parameters can be greater than 1, greater than 2, greater than 4, greater than 10, or greater than 100. The total number (n) of parameters may depend on the type of biomechanical activity to be evaluated and/or the physical condition of the human or animal subject. For example, n can be 50 or more when evaluating a basketball shot.

In the example of TABLE I, n=3, m=4, and processor device 16 computes A₁ for parameter P₁, A₂ for parameter P₂, and A₃ for parameter P₃. First quantity Aᵢ is computed by processor device 16 according to the following equation.

Aᵢ=min(Z, Vᵢ/Xᵢ) (Eq. 1A)

Variation vᵢ is as described above, and xᵢ is a maximum expected value of variation vᵢ. Factor z is a constant, which can be equal to 0.5, 1, 1.5, 2 or other number.

In the example of TABLE I, variation vᵢ for parameter P₁ would be a quantitative indicator of variation of 2.1, 2.4, 2.1, and 2.1 from a target value of P₁. If variation vᵢ is defined in processor device 16 as variance σᵢ², then the variation for parameter P₁ across four performances of the biomechanical activity would be the variance of 2.1, 2.4, 2.1 and 2.1. The variances for P₁, P₂ and P₃ are shown in TABLE I, which were computed using the average values of Pᵢ as the target for Pᵢ and assuming a total population of four performances of the biomechanical activity.

The subject may perform the biomechanical activity in a manner that results in a variance for a particular parameter greater than the expected maximum variance xᵢ for that parameter. Advantageously, the min( ) function could place a limit on the influence that parameter has on the consistency metric so as not unduly dominate or overshadow parameters having low variances. By definition, the min( ) function returns the lowest value contained in the array. For example, z may be defined in processor device 16 as equal to 1, and vᵢ may be defined in processor device 16 as σᵢ². Thus, equation 1A becomes:

Aᵢ=min(1, σᵢ²/xᵢ) (Eq. 1B)

In equation 1B, Aᵢ=σᵢ²/xᵢ when σᵢ² is less than xᵢ. Also, Aᵢ=1 when σᵢ² is greater than or equal to xᵢ.

Processor device 16 may compute the consistency metric by applying a second quantity (Bᵢ) to first quantity Aᵢ. Second quantity Bᵢ represents a level of influence parameter Pᵢ has on the consistency metric. Second quantity Bᵢ can be applied to first quantity Aᵢ by dividing or multiplying Aᵢ by Bᵢ to obtain third quantity Cᵢ. Alternatively, second quantity Bᵢ can be applied to first quantity Aᵢ by adding (or subtracting) Bᵢ to (or from) Aᵢ to obtain third quantity Cᵢ. Processor device 16 may compute the sum of all Cᵢ for inclusion in the consistency metric. The sum is computed according to according to Σᵢ₌₁ⁿCᵢ.

As indicated above, the biomechanical activity may include a series of biomechanical movements, and each movement may be characterized by one or more parameters. The consistency metric could be thought of as summarizing or synthesizing all the parameters of the biomechanical activity across several performances of the activity. However, some of the parameters could be more important than others. Advantageously, second quantity Bᵢ can be used to give priority or greater weight to more important parameters.

Additionally or alternatively, processor device 16 computes second quantity Bᵢ according to the following equation.

Bᵢ=kᵢ(c/n) (Eq. 2)

Factor c is a maximum possible value for the consistency metric. Factor c is a constant and can be set to any convenient number, such as 10 or 100. Other values for c can be used. The variable kᵢ is a weight factor for parameter Pᵢ. Weight factor kᵢ is useful in giving priority or greater weight to more important parameters. The weight factor for each parameter may be predefined based on human expertise, such as the expertise of an athletic coach, physical therapist, and/or biomechanics specialist. The weight factors may be stored in memory of processor device 16 or communicated to processor device 16 from device 22 (FIG. 1).

Additionally or alternatively, processor device 16 computes the consistency metric (CM) according to the following equation.

CM=c−Σᵢ₌₁ⁿ[Aᵢ×Bᵢ] (Eq. 3)

Again, c is the maximum possible value for the consistency metric. The consistency metric (CM) is likely to increase toward c when the human or animal subject improves by repeatedly performing the biomechanical activity such that the variance σᵢ² for many parameters are lower than the maximum expected variance xᵢ. Whether the consistency metric (CM) actually increases toward c may also depend on weight factors kᵢ.

When z is defined in processor device 16 as equal to 1, processor device 16 may compute the consistency metric (CM) as follows.

CM=c−Σᵢ₌₁ⁿ[kᵢ(c/n)×min(1, vᵢ/xᵢ)]

Again, variation vᵢ is a quantitative indicator of variation of values for a particular parameter Pᵢ across multiple performances of the biomechanical activity. As discussed above, vᵢ is the variation of parameter values from a target value, which may be a predefined in processor device 16 (e.g., based on a setting entered by a coach) or an average of parameter values.

When z is defined in processor device 16 as equal to 1, and vᵢ is defined in processor device 16 as a statistical variance, processor device 16 may compute the consistency metric (CM) as follows.

CM=c−Σᵢ₌₁ⁿ[kᵢ(c/n)×min(1, σᵢ²/xᵢ)]

For day to day use, sensor placement and calibration are important issues. It is extremely difficult to ensure identical placement of measurement equipment on or around a body of the subject across different training or therapy sessions. As used herein, a session refers to a period of time during which biomechanical data 14 and/or biometric data 20 are collected while the human or animal subject is repeatedly performing the biomechanical activity which is to be evaluated for consistency. A training or therapy session may take place on one day, and the next session may occur in the same day or on another day. Sessions may be separated by minutes, hours, days, weeks, or months. Usually it will not be feasible to place motion capture sensors 12 and/or biometric sensors 18 at precisely the same locations for all sessions. This can make it difficult to ascertain an improvement in consistency when comparing various sessions. It may be desirable to gather comparable measurements to enable computation of a single consistency metric associated with multiple sessions.

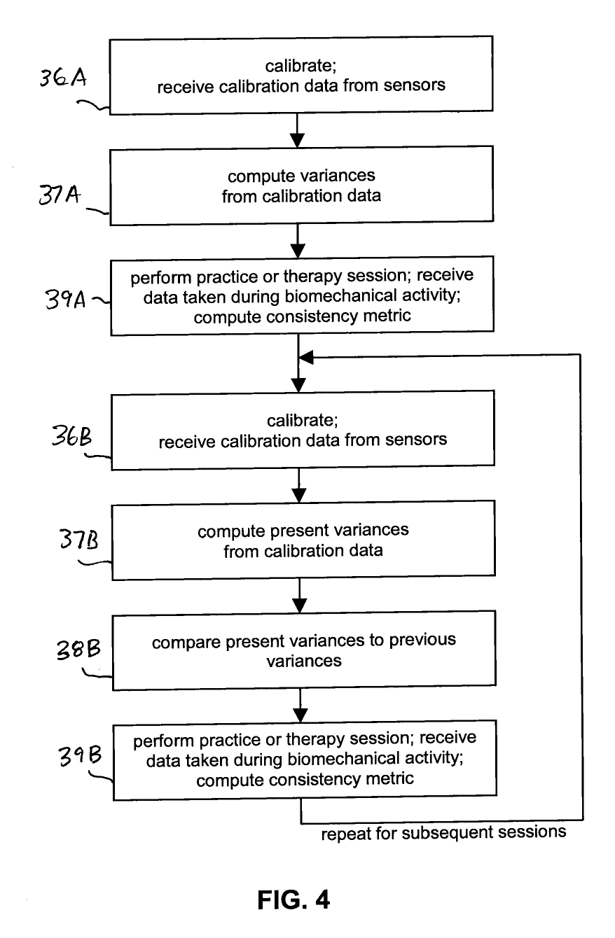

The effect of variability in placement of measurement equipment (motion capture sensors 12 and/or biometric sensors 18) may be reduced in several ways. One way is to calibrate sensor data at the start of the training or therapy session, as shown in FIG. 4. The calibration can ensure that the variance due to calibration is sufficiently bounded to enable processor device 16 to compute variances across measurements taken during different sessions.

After measurement equipment has been setup and put in place, the subject (such as an athlete or patient) calibrates system 10 before performing the actual biomechanical activity that is to be evaluated for consistency. Calibration at block 36A is accomplished by performing a series of predefined calibration movements prior to the practice or therapy session at block 39A. The calibration movements can be performed either by the subject or automatically on the measurement equipment. The calibration movements are measured by motion capture sensors 12. Variances in the measurements during calibration are computed by processor device 16 at block 37A.

Prior to starting another session at block 39B, calibration is performed again at block 36B with the same predefined movements as the previously calibration at block 36A. Variances in the measurements during calibration are computed by processor device 16 at block 37B. At block 38B, processor device 16 compares the present variances from block 37B to the previous variances from block 37A to determine whether they are comparable. The previous variances may be stored in memory of processor device 16 or communicated to processor device 16 from device 22.

For example, processor device 16 may check whether the present variances satisfy a similarity requirement relative to the previous variances. A similarity requirement could be that the present variances must be from 90% to 110% of the previous variances. Other types of similarity requirement can be used. The similarity requirement may be stored in memory of processor device 16 or communicated to processor device 16 from device 22. If the present variances satisfy the similarity requirement, then the consistency metric for the first session at block 39A can be compared, combined, or averaged with the consistency metric for the second session at block 39B.

If the present variances fail to satisfy the similarity requirement, then the user of system 10 may be prompted by processor device 16 to adjust or make corrections to the placement of the measurement equipment.

The process of calibrating, computing present variances from calibration movements, and comparing present variances to previous variances are repeated for each subsequent session.

Another way to reduce the effect of variability of measurements is to calibrate the sensor data during or after measurements of the biomechanical activity. This method assumes that the subject already has (prior to the session) baseline measurements for the biomechanical activity that is being evaluated for consistency. During each consecutive session, processor device 16 adjusts the values of the parameters. Values for parameters are determined from biomechanical data 14 and/or biometric data 20, and then the values are adjusted. Processor device 16 may make the adjustments by shifting individual values upward or downward to better match the distribution of the baseline measurements.

For example, processor device 16 may make the adjustments by finding an offset for individual values based on a baseline measurement statistic (e.g., median or average) and applying the offset to the values. The statistic may be stored in memory of processor device 16 or communicated to processor device 16 from device 22. For instance, processor device 16 may compute a median value of 58 for a particular parameter from baseline measurements, compute a median value of 88 for the same parameter from measurements taken during the session, and then subtract 30 (i.e., apply a downward offset of 30) from the individual values for the parameter when computing the consistency metric.

Processor device 16 may make the adjustments by using one or more values of parameters to compute an offset that is applied to the values to obtain adjusted values that match values of corresponding parameters from prior sessions. The values of corresponding parameters from prior sessions may be stored in memory of processor device 16 or communicated to processor device 16 from device 22.

Another way to reduce the effect of variability of measurements is for processor device 16 to compute a weighted average of consistency metrics across sessions. First, processor device 16 may calculate a consistency metric (CM) for each training or therapy session. Processor device 16 may compute CM1 for a first session, CM2 for a second session, and CM3 for a third session, and so on. Then processor device 16 may compute the weighted average as (W1×CM1)+(W2×CM2)+(W3×CM3)+ . . . , where the sum of all weights (W1+W2+W3 . . . ) equals 1 or 100%. The weight applied to each consistency metric can be influence by any or a combination of the following factors: the total number of performances of the biomechanical activity in the session; the confidence in the quality of the data 14 and/or data 20 obtained during the session; recency of a session (whether the session occurred recently or a long time ago); whether an expert, e.g., coach or therapist, was present during the session; and biometric factors, e.g., fatigue or other bodily conditions and health indicators, which may be determined from biometric data 20.

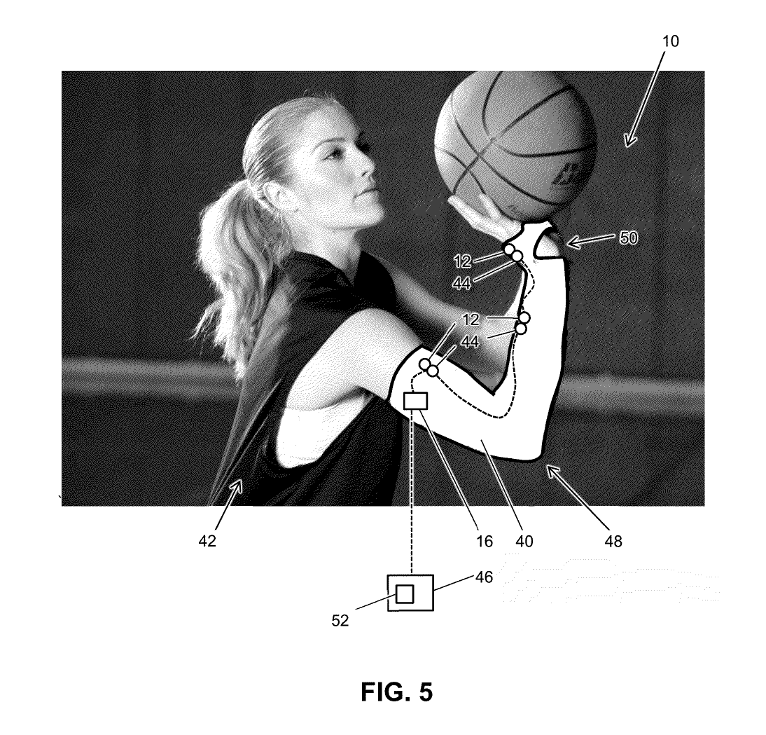

In FIG. 5, system 10 includes sensors 12 that are mounted on fabric sleeve 40 which can be worn while playing a sport such as basketball. Subject 42 is an athlete. System 10, which is in the form of a training sleeve, can provide a basketball player or other person with information as to whether a particular athletic skill, such as a basketball shot or other biomechanical activity, is being performed consistently. In addition, system 10 can provide feedback as to whether the biomechanical activity was performed with good biomechanical form.

Sensors 12 detect the primary shooting arm of athlete 42. Sleeve 40 mounts sensors 12 to the arm of athlete 42. Sensors 12 enable processor device 16 to detect when athlete 42 attempts a basketball shot (as opposed to another maneuver, such as dribbling the basketball ball) and to analyze the biomechanics of the basketball shot. Athlete 42 can receive immediate feedback through audio and visual indicators produced by feedback devices 44 coupled to sensors 12.

Feedback devices 44 may include lights (e.g., light emitting diodes or lamps) and/or speakers or other device configured to generate a sound. When the athlete's form is incorrect or undesirable, feedback devices 44 emit a light and/or sound to indicate how to improve future basketball shot. Athlete 42 may also track her performance and compare it to that of teammates using a software application program running on mobile device 46 communicatively coupled to processor device 16. Examples for mobile device 46 include without limitation a smartphone, tablet computer, and laptop computer. Mobile device 46 can be owned or operated by athlete 42 or another person.

Training sleeve 10 includes three motional capture sensors 12: one on the back of the hand, one on the forearm, and one on the upper arm. Each sensor 12 includes a 3-axis accelerometer, a 3-axis gyroscope, and a 3-axis compass which, in combination, accurately track rotation and motion in space using algorithms. Sensors 12 are communicatively coupled to processor device 16 which applies the algorithms to sensor data 14. Sensors 12 are sampled by processor device 16 at around 200 times per second. From sensor data 14, processor device 16 can determine the current rotation of the shoulder, elbow, and wrist, and thereby determine values for parameters associated with the shoulder, elbow, and wrist.

Sensors 12 are located on opposite sides of elbow joint 48 and wrist joint 50. This arrangement allows processor device 16 to determine the angles of the elbow and wrist during various biomechanical events (e.g., start of biomechanical movement, and a momentary pause in movement between the end of one biomechanical movement and the start of another biomechanical movement) and during various biomechanical movements. Also, this arrangement allows processor device 16 to measure the rate of rotational movement of the upper arm, forearm, and wrist.

Processor device 16 uses data 14 from sensors 12 to compute a consistency metric. This may be accomplished using algorithms running in processor device 16. The basketball shot is broken down into many measurable discrete parameters, and biomechanical data 14 is used to obtain values for the parameters, which may include without limitation joint angles, acceleration, rotation, and direction of movement. The values for the parameters are used to compute the consistency metric. Processor device 16 may display the consistency metric on a display screen of the mobile device 46.

In addition, processor device 16 may compare the parameter values to requirements for good form defined in a model. The requirements contained in the model can be configured or modified by athlete 42 or other person using the software application program running on mobile device 46 and input device 52 (such as a touch sensitive screen or keyboard) of mobile device 46.

Biometric sensors may also be mounted on fabric sleeve 40 to enable processor device 16 of training sleeve 10 to characterize any one or a combination of bodily conditions (e.g., muscle activity, muscle fatigue, heart rate, etc.) previously described. Thus, the parameters used by processor device 16 of training sleeve 10 may include a parameter that defines one or more bodily conditions.

Processor device 16 can communicate with mobile device 46 using Bluetooth or other wireless, over-the-air communication protocol. This can allow all sensor data 14 from training sleeve 10 to be uploaded by mobile device 46 to a cloud storage environment. A cloud storage environment refers to storage of data in any number of computer servers at any number of physical locations, and the computer servers are owned and managed, not by the individual using training sleeve 10, but by a hosting company.

Analysis such as computing a consistency metric for each practice session, computing trends in consistency metrics of consecutive training sessions and/or computing a single consistency metric for several training sessions, can be performed either on mobile device 46, in the cloud, or both. Mobile device 46 can also be used to personalize settings for one or more athletes, as well as to update the software and algorithms running on processor device 16. For example, input device 52 can be used to enter values that affect Bᵢ, kᵢ, xᵢ, c, W1, W2, W3, and/or other factors.

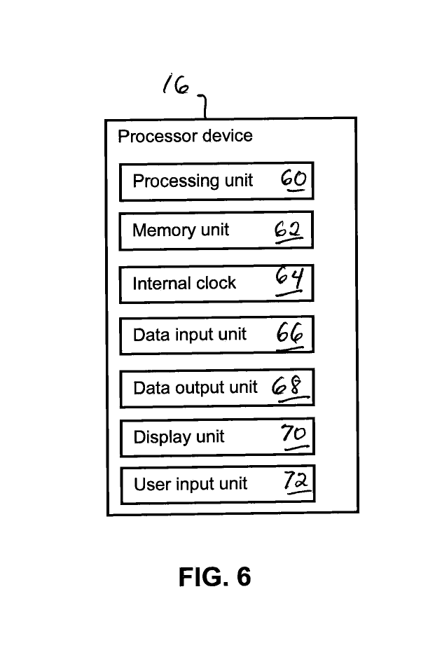

In any of the aspects described in association with FIGS. 1-5, processor device 16 may include various components as shown in FIG. 6. In FIG. 6, exemplary processor device 16 includes processing unit 60 that analyzes data received from motion capture sensors 12 and/or biometric sensors 18. Although processor device 16 is schematically depicted as a single box, it should be understood that various components of processor device 16 can be housed together in a single case or can be housed in separate cases while still being communicatively coupled with each other.

Processing unit 60 may include one or more circuit assemblies, microprocessors and electronic semiconductor chips. Memory unit 62 includes one or more memory components, e.g., components for volatile and/or non-volatile data storage, for storing data received from motion capture sensors 12 and/or biometric sensors 18. Internal clock 64 enables processor device 16 to determine timing of biomechanical movements. Data receiver unit 66 is configured to receive data from motion capture sensors 12 and/or biometric sensors 18. Data receiver unit 66 may include various components (e.g., antennas, electrical connectors, and data processing circuitry) that allow the data to be received wirelessly through the air (e.g., via radio signals or other electromagnetic radiation in the air) or by wire (e.g., electrical or fiber optic cable).

Optionally, processor device 16 may also include data output unit 68 that enables processor device 16 to export data to another device, such as mobile device 46 (FIG. 5). Data output unit 68 may include various components (e.g., antennas, electrical connectors, and data processing circuitry) that allow sensor data or results of data analysis (e.g., consistency metrics) to be transmitted wirelessly or by wire. Data output unit 68 may also handle transmission of signals to feedback devices 44 (FIG. 5). Processor device 16 may also include display unit 70 that enables processor device 16 to visually display text and/or graphics that represent sensor data, consistency metrics, and other results of data analysis. Display unit 70 can be a liquid crystal display screen, light emitting diode display screen, or other type of electronic display. Processor device 16 may also include user input unit 72 that allows a person to enter values that affect Bᵢ, kᵢ, xᵢ, c, W1, W2, W3, and/or other factors. The person may also use input unit 72 to enter settings that prescribe the parameters to be measured, and/or enter adjustments to requirements contained in a model of a biomechanical activity. Input unit 72 can be a keyboard, touch sensitive screen, microphone, or a remote control button.

Processor device 16 can be capable of executing, in accordance with a computer program stored on a non-transitory computer readable medium, any one or a combination of the steps and functions described above for receiving sensor data from motion capture sensors 12 and/or biometric sensors 18, determining parameter values that quantify a biomechanical activity, and using the values to compute a consistency metric. The non-transitory computer readable medium may comprise instructions for performing any one or a combination of the steps and functions described herein. Processor device 16 (optionally memory unit 62) may include the non-transitory computer readable medium. Examples of a non-transitory computer readable medium includes without limitation non-volatile memory such as read only memory (ROM), programmable read only memory, and erasable read only memory; volatile memory such as random access memory; optical storage devices such as compact discs (CDs) and digital versatile discs (DVDs); and magnetic storage devices such as hard disk drives and floppy disk drives.

In any of the aspects described in association with FIGS. 1-6, motion capture sensors 12 may include an inertial measurement unit (IMU), which is a type of motion sensor. The IMU is configured to detect motion of the body. The IMU can be the ones described in U.S. Patent Application Publication No. 2014/0150521 (titled “System and Method for Calibrating Inertial Measurement Units”). An IMU is configured to provide information on its orientation, velocity, and acceleration. An IMU may include gyroscopes, accelerometers, and/or magnetometers. A gyroscope is configured to measure the rate and direction of rotation. An accelerometer is configured to measure linear acceleration. A magnetometer (a type of compass) is configured to detect direction relative to magnetic north pole.

As previously mentioned, biometric sensors 18 may also include myography sensors configured to detect whether a particular muscle is being used by the person and optionally how fatigued that muscle is. Myography sensors include sensors configured to provide signals indicative of muscle contraction, such as signals corresponding to electrical impulses from the muscle, signals corresponding to vibrations from the muscle, and/or signals corresponding to acoustics from the muscle, as described in U.S. Patent Application Publication No. 2014/0163412 (titled “Myography Method and System”). Other exemplary myography sensors include those described in U.S. Patent Application Publication Nos. 2010/0262042 (titled “Acoustic Myography Systems and Methods”), 2010/0268080 (titled “Apparatus and Technique to Inspect Muscle Function”), 2012/0157886 (titled “Mechanomyography Signal Input Device, Human-Machine Operating System and Identification Method Thereof”), 2012/0188158 (titled “Wearable Electromyography-based Human-Computer Interface), 2013/0072811 (titled “Neural Monitoring System”), and 2013/0289434 (titled “Device for Measuring and Analyzing Electromyography Signals”).

Myography sensors include without limitation a receiver device configured to detect energy which has passed through the person's body or reflected from the person's body after having been transmitted by a transmitter device. The receiver device need not be in contact with the person's skin. Myography sensors with these types of receiver and transmitter devices are described in co-pending application Ser. No. 14/506,322 (titled “Myography Method and System”), filed Oct. 3, 2014. The type of energy transmitted by the transmitter device and then received by the receiver device includes without limitation sound energy, electromagnetic energy, or a combination thereof, which are used to infer vibrations occurring on the skin surface, below the skin surface, or in the muscle which naturally arise from muscle contraction. For example, the transmitter device can be configured to transmit (and receiver device can be configured to detect) audio signals, which may include acoustic waves, ultrasonic waves, or both. Acoustic waves are in the range of 20 Hz to 20 kHz and include frequencies audible to humans. Ultrasonic waves have frequencies greater than 20 kHz. Additionally or alternatively, transmitter can be configured to transmit (and receiver 16 can be configured to detect) radio waves. For example, radio waves can have frequencies from 300 GHz to as low as 3 kHz. Additionally or alternatively, the transmitter device can be configured to transmit (and receiver device can be configured to detect) infrared light or other frequencies of light. For example, infrared light can have frequencies in the range of 700 nm to 1 mm. These types of energy, after having passed through the person's body or reflected from the person's body, are analyzed by processor device 16 to infer muscle contraction and/or muscle fatigue.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

1. A method for assessing consistency of performance of a biomechanical activity, the method comprising:
receiving data from sensors configured to detect a biomechanical activity of a body, the data representing multiple performances of the biomechanical activity;
for each performance of the biomechanical activity, determining a value for each one of a plurality of parameters that quantify various aspects of the biomechanical activity, the value being determined from the received data; and
computing a consistency metric representing biomechanical similarity of the multiple performances of the biomechanical activity, the computing performed using the values for the plurality of parameters from the multiple performances of the biomechanical activity.

2. The method of claim 1, wherein the plurality of parameters includes a parameter that defines a position or orientation of a part of the body, and the position or orientation is either (a) an absolute position or orientation in three-dimensional space, (b) a relative position or orientation with respect to another part of the body, or (c) a relative position or orientation with respect to an object that is not part of the body.

3. The method of claim 1, wherein the biomechanical activity includes a plurality of biomechanical movements, and the plurality of parameters includes a parameter that defines timing of one of the biomechanical movements relative to another one of the biomechanical movements.

4. The method of claim 1, wherein the plurality of parameters includes a parameter that defines muscle activity before, during, or after one or more biomechanical movements of the biomechanical activity.

5. The method of claim 1, wherein the plurality of parameters includes a parameter that defines muscle fatigue before, during, or after one or more biomechanical movements of the biomechanical activity.

6. The method of claim 1, wherein the plurality of parameters includes a parameter that defines a bodily condition before, during, or after one or more biomechanical movements of the biomechanical activity, and the bodily condition is any one of heart rate, heart rate variability, skin conductance, blood oxygen level, blood sugar level, respiration rate, and neurological activity.

7. The method of claim 1, wherein computing the consistency metric includes computing a first quantity (Aᵢ) for each of the plurality of parameters Pᵢ with i=1 to n and n≧2, the first quantity (Aᵢ) representing a level of variation in the values of the parameter Pᵢ across the multiple performances of the biomechanical activity, the first quantity (Aᵢ) computed according to
Aᵢ=min(1, vᵢ/xᵢ)
wherein vᵢ is a variation of values of the parameter Pᵢ across the multiple performances of the biomechanical activity, and xᵢ is a maximum expected value of vᵢ.

8. The method of claim 7, wherein computing the consistency metric includes applying, for each of the plurality of parameters, a second quantity (Bᵢ) to the first quantity (Aᵢ) to produce a third quantity (Cᵢ), wherein B, represents a level of influence parameter Pᵢ has on the consistency metric, and computing the consistency metric further includes computing a sum according to Σᵢ₌₁ⁿCᵢ.

9. The method of claim 8, wherein computing the consistency metric includes computing, for each of the plurality of parameters, the second quantity (Bᵢ) according to
Bᵢ=kᵢ(c/n)
wherein c is a maximum possible value for the consistency metric, kᵢ is a weight factor for parameter Pᵢ, and computing the consistency metric (CM) is performed according to
CM=c−Σᵢ₌₁ⁿ[Aᵢ×Bᵢ].

10. The method of claim 1, the method further comprising receiving calibration data from the sensors, computing present variances from the calibration data, comparing the present variances to previous variances that were computed from previously received calibration data to determine whether the present variances satisfy a similarity requirement to the previous variances.

11. The method of claim 1, wherein computing the consistency metric includes:
using one or more values of the plurality of parameters to compute an offset that, when applied to the one or more values, causes the one or more values to match one or more previous values of the same parameters, the previous values associated with previous performances of the biomechanical activity;
applying the offset to the values for the plurality of parameters from the present performances of the biomechanical activity to obtain adjusted values; and
using the adjust values to compute the consistency metric.

12. The method of claim 1, using the computed consistency metric, which corresponds to present performances of the biomechanical activity, to obtain a weighted average consistency metric that corresponds to previous performances of the biomechanical activity and the present performances of the biomechanical activity.

13. The method of claim 1, wherein at least one of the sensors includes a camera, and a computer device is used to determine the values for the parameters from the received data.

14. The method of claim 1, wherein at least one of the sensors is worn on the body.

15. A system for assessing consistency of performance of a biomechanical activity, the system comprising:
a plurality of sensors configured to detect a biomechanical activity of a body, the plurality of sensors configured to provide data on multiple performances of the biomechanical activity; and
a processor device configured to receive and use the data to determine, for each performance of the biomechanical activity, a value for each one of a plurality of parameters that quantify various aspects of the biomechanical activity, the processor device further configured to use the values for the plurality of parameters from the multiple performances of the biomechanical activity to compute a consistency metric representing biomechanical similarity of the multiple performances of the biomechanical activity.

16. The system of claim 15, wherein each of the values determined by the processor device for at least one of the parameters represents a position or orientation of a part of the body, and the position or orientation is either (a) an absolute position or orientation in three-dimensional space, (b) a relative position or orientation with respect to another part of the body, or (c) a relative position or orientation with respect to an object that is not part of the body.

17. The system of claim 15, wherein the biomechanical activity includes a plurality of biomechanical movements, and the value determined by the processor device for at least one of the parameters represents timing of one of the biomechanical movements relative to another one of the biomechanical movements.

18. The system of claim 15, wherein at least one of the sensors is a myography sensor, and the value determined by the processor device for at least one of the parameters represents muscle activity before, during, or after one or more biomechanical movements of the biomechanical activity.

19. The system of claim 15, wherein the value determined by the processor device for at least one of the parameters represents muscle fatigue before, during, or after one or more biomechanical movements of the biomechanical activity.

20.-25. (canceled)

26. A non-transitory computer readable medium having a stored computer program embodying instructions, which when executed by a computer system, causes the computer system to provide an assessment of consistency of performance of a biomechanical activity, the computer readable medium comprising:
instructions for receiving data from sensors configured to detect a biomechanical activity of a body, the data representing multiple performances of the biomechanical activity;
instructions to determine, for each performance of the biomechanical activity, a value for each one of a plurality of parameters that quantify various aspects of the biomechanical activity, the value being determined from the received data; and
instructions for computing a consistency metric representing biomechanical similarity of the multiple performances of the biomechanical activity, the computing performed using the values for the plurality of parameters from the multiple performances of the biomechanical activity.

27. (canceled)
